(19) United States
(12) Patent Application Publication
(10) Pub. No.: US 2006/0121062 A1
(43) Pub. Date: Jun. 8, 2006

(54) Conjugated hydroxyalkyl starch allergen compounds

(76) Inventors: Wolfram Eichner, Butzbach (DE); Dirk Dormann, Mainz-Kostheim (DE)

Correspondence Address: FISH & RICHARDSON P.C., PO BOX 1022, MINNEAPOLIS, MN, 55440-1022, US

(21) Appl. No.: 10/527,554

(22) PCT Filed: Sep. 2, 2003
(86) PCT No.: PCT/EP03/09750

§ 371 Date: Jul. 26, 2005

(30) Foreign Application Priority Data
Sep. 11, 2002 (DE) 102 42 076.9

(51) Int. Cl. A61K 39/35; C07K 14/415

(52) U.S. Cl. 424/275.1; 530/395

(57) ABSTRACT

The invention relates to a conjugated compound of hydroxyalkyl starch and an allergen, in which at least one hydroxyalkyl starch is covalently coupled to the allergen.

The present invention relates to compounds which comprise a conjugate of a hydroxyalkylstarch (HAS) and an allergen, where the HAS is covalently linked either directly or via a linker to the allergen. The invention further relates to processes for preparing corresponding conjugates and to the use thereof as medicaments.

### TECHNICAL BACKGROUND

Excessive specific reactions of the immune system against exogenous substances are nowadays encompassed by the term allergies. According to the classification of Coombs and Gell, allergic reactions can be categorized into types I to IV which can be differentiated inter alia on the basis of the classes of antibody involved in the reaction, of the antigens recognized and of the induced effector mechanisms.

Compounds referred to as allergens are accordingly those able to induce an allergic immune response, in the narrower sense an immediate-type allergic immune response (type I), on the skin and mucosa. The allergens are normally polypeptides or proteins with a molecular weight of about 5000 to about 80 000 Da. The polypeptides may be of vegetable, animal or microbiological origin. The polypeptides may additionally be present as constituents of house dust.

Allergens induce IgE antibodies which bind by their constant part to the surface of mast cells and thus bring about degranulation of the mast cells. The substances released by mast cells (histamines, proteolytic enzymes and inflammatory mediators) cause directly and indirectly the symptoms of an allergy, normally rhinitis, conjunctivitis and/or bronchial asthma.

IgE-mediated immediate-type allergens (type I) are the form of allergic reactions with by far the greatest prevalence. Up to 20% of people in industrialized countries suffer from type I allergic symptoms. Allergy sufferers are currently treated in addition to pharmacotherapy by specific immunotherapy, called hyposensitization (Kleine-Tebbe et al., Pneumologie, Vol. 5 (2001), 438-444).

In conventional hyposensitization, a specific allergen extract is administered subcutaneously in increasing quantities until an individual maintenance dose is reached. As the treatment is continued, this dose is administered repeatedly, various treatment protocols being employed (Klimek et al., Allergologie und Umweltmedizin, Schattauer Verlag, page 158 et seq.).

The result of therapy in this case appears to be closely connected with the quantities of allergen employed during the maintenance phase. If the administered quantities of allergen are increased, however, the risk of an IgE-mediated reaction of the allergic patient is also always increased. In other words, use of the therapy is also restricted by the allergic reaction of the patient and the risk associated therewith for the patient of anaphylactic shock.

The therapy is regarded as successful if the allergic symptoms are reduced, leading to an individual decline in the requirement for medicines and an increase in the tolerance of the allergen.

It has already been proposed that some allergenic polypeptides be generated by recombinant expression and be used for hyposensitization (DE 100 41 541).

In order to obtain allergens with reduced IgE-binding properties, they have been modified with polyethylene glycol (PEG) and used for the hyposensitization. A large number of publications accordingly describes the preparation of PEG-allergen conjugates which were generated by covalent bonding of an allergen to polyethylene glycol. Mosbech et al. (Allergy, 1990, Vol. 45(2): 130-141) report for example the treatment Of allergic adults with asthma using PEG-house dust conjugates and the immunological response after the treatment. The authors found a clinical improvement of the effect as long as the dosage of the allergen was sufficient to reduce the amount of specific IgE and/or to induce IgG, in particular IgG4, responses.

Similarly, Schafer et al. (Ann. Allergie, 1992, Vol. 68(4): 334-339) report on a study in which an allergenic composition of a PEG-modified grass pollen mix was used for hyposensitization of adults. The results were compared with those obtained by hyposensitization using the partly purified grass pollen mix. The treatment took place in a double-blind study. The frequency and the extent of the side effects were reduced by about 50% by PEG modification. A significant improvement in the hypersensitivity was found in both treatment groups.

PEG conjugates do not, however, have any naturally occurring structure for which in vivo degradation pathways have been described.

Besides PEG conjugates, other allergen derivatives have also been prepared and investigated. Thus, dextran-modified allergens generated by conjugation with carboxymethyldextran are known. Some studies with beta-lactoglobulin have shown that the antibody response to dextran conjugates is considerably attenuated by comparison with unmodified compounds (Kobayashi et al., J Agric Food Chem 2001 February; 49(2): 823-31; Hattori et al., Bioconjug Chem 2000 January-February; 11(1): 84-93).

In addition, crosslinked high molecular weight allergens, called allergoids, have been generated. It was possible to obtain these products for example by formaldehyde or glutaraldehyde modification of allergens. Corresponding products can be obtained from Allergopharma, Joachim Ganser KG, 21462 Reinbek; HAL Allergie GmbH, 40554 Düsseldorf; and SmithKline Beecham Pharma GmbH, Benckard, 80716 Munich.

A comprehensive review of the scope of various processes for preparing bioconjugates in general is given by G. T. Hermanson (Bioconjugate Techniques, Academic Press, San Diego 1996). In this context, linkage of oligo- and polysaccharides to proteins mostly takes place via lysine (—NH₂) or cysteine (—SH) side chains and less commonly via aspartic or glutamic acid (—COOH) or else tyrosine (aryl-OH) side chains. However, to date, starch derivatives have not been used to modify allergens.

Hydroxyethylstarch for example is a substituted derivative of the carbohydrate polymer amylopectin which constitutes 95% of corn starch. HES has advantageous Theological properties and is currently employed clinically for volume replacement and for hemodilution therapy (Sommermeyer et al., Krankenhauspharmazie, Vol. 8(8), (1987), pp. 271-278; and Weidler et al., Arzneim.-Forschung/Drug Res., 41, (1991) 494-498).

Amylopectin consists of glucose units, with α-1,4-glycosidic linkages being present in the main chains but α-1,6-glycosidic linkages at the branch points. The physicochemical properties of this molecule are essentially determined by the nature of the glycosidic linkages. Owing to the angulated α-1,4-glycosidic linkage, helical structures with about 6 glucose monomers per turn are formed.

The physicochemical and the biochemical properties of the HES polymer can be modified by substitution. Introduction of a hydroxyethyl group can be achieved by alkaline hydroxyethylation. It is possible through the reaction conditions to exploit the difference in reactivity of the particular hydroxyl group in the unsubstituted glucose monomer towards hydroxyethylation, thus making it possible to influence the substitution pattern.

HES is therefore essentially characterized by the molecular weight distribution and the level of substitution. The level of substitution can in this connection be described by the DS (“degree of substitution”) which refers to the substituted glucose monomers as a proportion of all the glucose units, or by the MS (“molar substitution”) which indicates the number of hydroxyethyl groups per glucose unit.

HES solutions are polydisperse compositions in which the individual molecules differ from one another in the degree of polymerization, the number and arrangement of the branch points and their substitution pattern. HES is thus a mixture of compounds differing in molecular weight. Accordingly, a particular HES solution is defined by an average molecular weight on the basis of statistical variables. In this connection, Mₙ is calculated as simple arithmetic mean as a function of the number of molecules (number average), while M_{w}, the weight average, represents the mass-dependent measured variable.

The present invention is thus based on the object of providing improved allergen derivatives, in particular allergen derivatives which achieve a depot effect and therefore need to be administered less often.

This object has now been achieved by conjugates of hydroxyalkylstarch (HAS) and allergen in which at least one hydroxyalkylstarch is covalently coupled to the allergen.

Accordingly, it has surprisingly been found according to the invention that the HAS-allergen-conjugates can be used particularly advantageously for specific immunotherapy. The safety of hyposensitization is increased by the use of the conjugates of the invention. At the same time, the conjugates of the invention have a higher in vivo half-life, and thus conjugation with HAS achieves a depot effect which has a beneficial influence on the clinical efficacy. The depot effect of the conjugates of the invention has the advantage, in particular compared with aqueous allergen extracts, that the conjugates need to be administered less frequently in order to achieve a therapeutic effect.

The HAS-allergen conjugates of the invention can be prepared in such a way that they show a reduced, compared with unmodified allergens, binding to allergen-specific IgE. The HAS-allergen conjugates can in a particularly preferred embodiment show only very low or absolutely no specific binding to allergen-specific IgE. The conjugates of the invention can thus be administered in higher dosage, in turn increasing the probability of successful hyposensitization.

Compared with crosslinked allergoids, the HAS-allergen conjugates of the present invention have the advantage that they can provide an epitope profile comparable to the natural allergen. The efficacy of immunotherapy can thus be increased. By contrast, the polymerization of allergens using formaldehyde or glutaraldehyde leads to poorly defined high molecular weight compounds (Crit Rev Ther Drug Carrier Syst 1990; 6(4): 315-65) which may generate unnatural epitopes, so that their effect would have to be investigated in the individual case.

In the conjugate, at least one hydroxyalkylstarch is coupled to an allergen. The scope of the invention also of course includes coupling products which comprise a plurality of hydroxyalkylstarch molecules and one allergen molecule or a plurality of allergen molecules and one hydroxyalkylstarch molecule.

The hydroxyalkylstarch may be present in the conjugate coupled directly to the allergen or via a linker to the allergen. The hydroxyalkylstarch may also be coupled to the polypeptide chain or to one or more of the saccharide chains of an allergenic glycoprotein.

Hydroxyalkylstarch (HAS)

The term “hydroxyalkylstarch” is used for the purposes of the present invention to refer to starch derivatives which have been substituted by a hydroxyalkyl group. The hydroxyalkyl group preferably includes 2 to 4 C atoms. The group referred to as “hydroxyalkylstarch” thus preferably comprises hydroxyethylstarch, hydroxypropylstarch and hydroxybutylstarch. The use of hydroxyethylstarch (HES) as coupling partner is particularly preferred for all embodiments of the invention.

It is preferred according to the invention for the hydroxyethylstarch employed to prepare the conjugates to have an average molecular weight (weight average) of 1-300 kDa, with an average molecular weight of from 5 to 200 kDa being particularly preferred. Hydroxyethylstarch may moreover have a level of molar substitution of 0.1-0.8 and a C₂:C₆ substitution ratio in the region of 2-20, in each case based on the hydroxyethyl groups.

Allergens

The compounds referred to as allergens for the purposes of the present invention are primarily those able to induce allergic immune responses, in the narrower sense IgE-mediated hypersensitivity reactions (type I). Also included are peptides derived from the sequence of an allergen, such as, for example, cleavage products resulting from enzymatic cleavages. Corresponding allergens are employed for specific immunotherapy and are commercially available.

Allergens can be isolated from natural sources. Thus, in the case of pollen allergens for example allergen extracts are obtained from the particular pollens. In addition, for example, recombinant preparation of the allergens is possible.

The allergens are preferably compounds selected from the group consisting of polypeptides, proteins, and glycoproteins.

### PREPARATION PROCESS

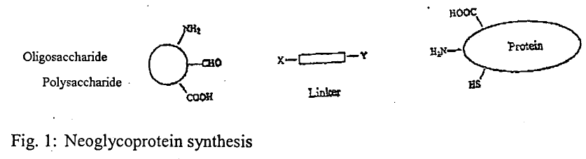

In one aspect, the invention relates to processes for preparing HAS-allergen conjugates in which HAS is covalently coupled either directly or via a linker to the allergen. The coupling can in this connection take place in various ways. A general structure of a neoglycoprotein synthesis using a linker is shown in FIG. 1.

In one embodiment, the present invention relates to processes for preparing HAS-allergen conjugates in which HES is linked to an ε-NH₂ group, to an α-NH₂ group, to an SH group, to a COOH group or to a —C(NH₂)₂ group of an allergen.

The invention also includes processes in which HES is coupled by reductive amination to the ε-NH₂ group of a protein. As alternative to this, the invention relates to processes in which the allergen is coupled to the reducing end groups of hydroxyethylstarch.

In a further embodiment, the invention relates to processes in which an active group is introduced into the HAS for the coupling to the allergen. The active group may be for example an aldehyde, thiol or an amino function.

The allergen and the oligo- or polysaccharide can be coupled together either directly or with use of a linker. It is possible to employ any crosslinker as linker. The linker may be for example a bifunctional linker or a homo- or heterobifunctional crosslinker.

Numerous crosslinkers such as, for example, SMCC (succinimidyl 4-(N-maleimidomethyl)cyclohexane-1-carboxylate) are commercially available and familiar to the skilled worker (cf. alphabetical list of “cross-linking reagents” in the Perbio product catalog and www.piercenet.com) and can be used for the purposes of the present invention.

The present invention relates in a further embodiment to the HAS-allergen conjugates obtainable by the processes described.

Some processes for synthesizing HAS-allergen conjugates are described generally below. The average skilled worker active in the bioconjugate sector will have no problems in selecting from the described processes those which are particularly suitable in relation to the objects to be achieved (chosen allergen, chosen HAS, etc.).

Direct Coupling of Unmodified HAS to Allergenic Proteins by Reductive Amination:

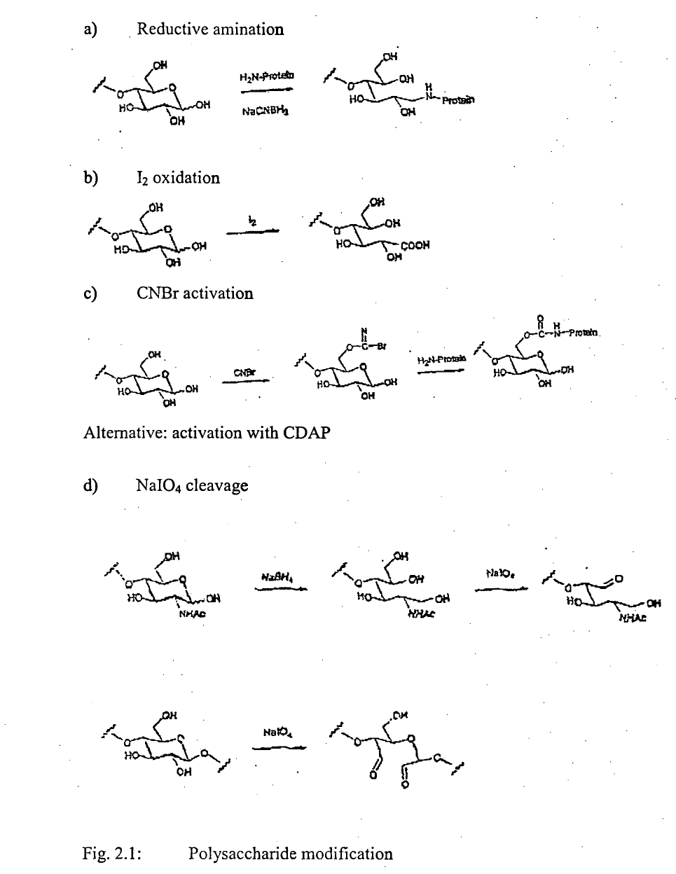

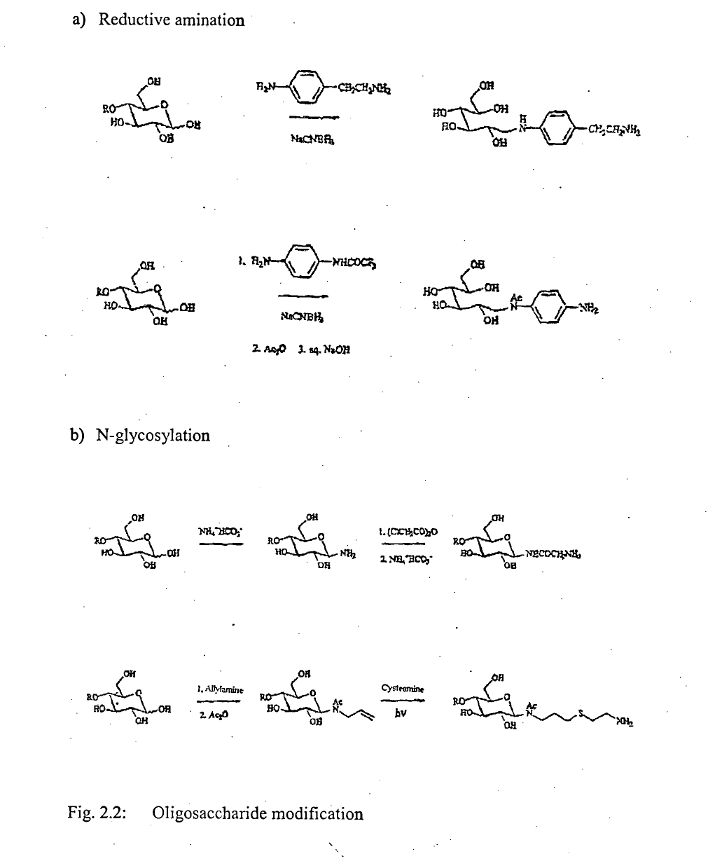

Direct coupling of the HAS to the ε-amino groups of the allergenic protein via a reductive amination in the presence of NaCN/BH₃ represents a simple and mild process which can be carried out without modifying the HAS (G. R. Gray, Arch. Biochem. Biophys. 1974, 163, 426-28) (FIG. 2.1a).

Reducing agents which can also be employed are pyridine-borane and other amino-borane complexes which are more stable and easier to handle (J. C. Cabacungan et al., Anal. Biochem. 1982, 124, 272-78). In contrast to an acylation, the modified amino group of the protein remains positively charged under physiological conditions. The effects on the tertiary structure of the protein are therefore less in the case of reductive amination. However, in this process the ring structure of the reducing sugar is lost.

Processes for Coupling Modified HAS:

Oxidation of the Reducing End to Aldonic Acids

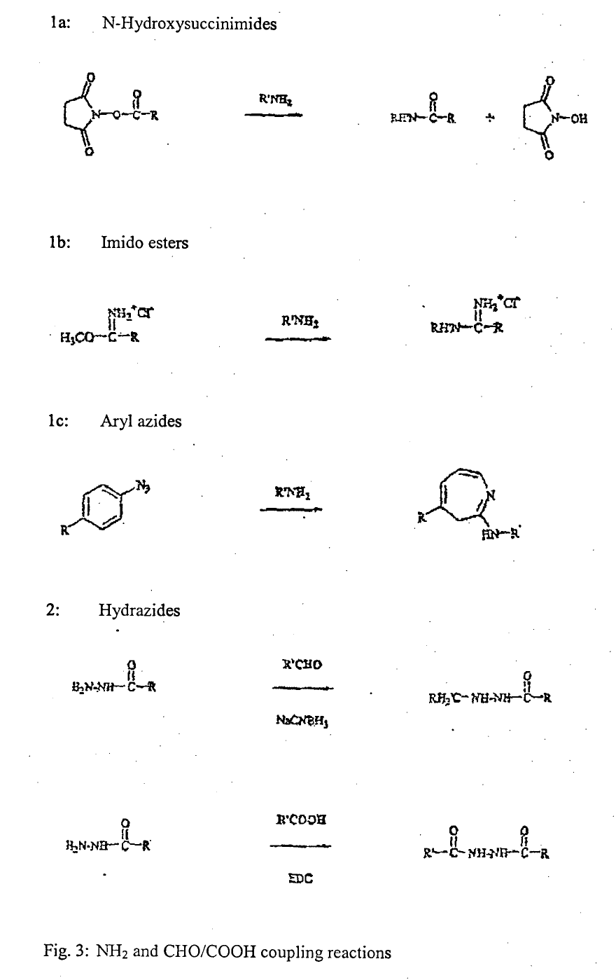

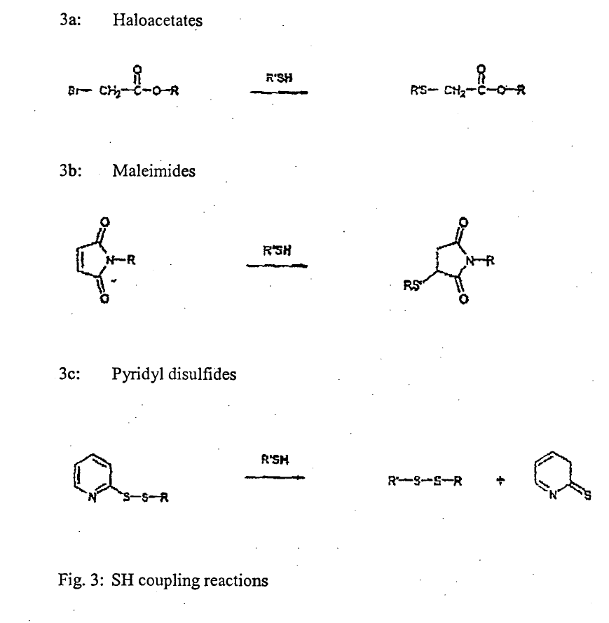

In the rarely used oxidation with iodine (or bromine) to the corresponding aldonic acid (G. Ashwell, Methods of Enzymol. 1972, 28, 219-22), the ring structure of the reducing sugar is lost (FIG. 2.1b), in addition careful control of the reaction is necessary in order to avoid nonspecific oxidation. The carboxylic acid function which is formed can be coupled in the presence of EDC (1-ethyl-3-(3-dimethylaminopropyl)carbodiimide) (J. Lönngren, I. J. Goldstein, Methods Enzymol. 1994, 247, 116-118) with the ε-amino groups of the lysine side chains of the allergenic protein or via a hydrazide linker (see FIG. 3). It is also possible to use the carboxyl groups present in the polysaccharide structures of, for example, mannuronic, glucuronic or sialic acids analogously for the coupling.

A particularly preferred embodiment of the present invention provides compounds which consist of an HES-allergen conjugate in which the allergen is specifically linked to the reducing end groups of the hydroxyethylstarch. For this purpose, the reducing end groups can previously be oxidized selectively, for example by the process described in Hashimoto et al. (Kunststoffe, Kautschuk, Fasern, Vol. 9, (1992), pp. 1271-1279) for oxidizing the reducing aldehyde end group of a saccharide.

Activation of the Hydroxy Function of the HAS

One of the most useful methods for nonspecific activation of polysaccharides is reaction with cyanogen bromide (CNBr) (C. Chu et al., Infect. Immun. 1983, 40, 245-56) (FIG. 2.1c). The activated hydroxy groups acylate lysine, cysteine and histidine side chains of the protein. This coupling process may, however, have disadvantages which are attributable to the high pH and to the toxicity and poor manageability.

An alternative to CNBr is provided by CDAP (1-cyano-4-dimethylaminopyridinium tetrafluoroborate) (A. Lees et al., Vaccine 1996, 14, 190-98; D. E. Shafer et al., Vaccine 2000, 18, 1273-81) which has increased reactivity of the cyano group and which makes reaction possible under very much milder conditions.

In general, unspecific activations of polysaccharides may lead to multiple substitution and thus also to crosslinking between polysaccharide and protein. However, this can be substantially suppressed through suitable choice of the reaction conditions.

Introduction of Aldehydes

Aldehyde functions can also be introduced into nonreducing polysaccharides by cleaving vicinal hydroxy groups with NaIO₄ (J. M. Bobbit, Ad. Carbohydr. Chem. 1956, 11, 1-41) (FIG. 2.1d), it being possible to achieve adequate selectivity via the concentration of the sodium periodate solution. Sialic acid is particularly easy to oxidize (S. M. Chamov et al., Biol. Chem. 1992, 267, 15916-22).

The reaction rate in the direct reductive amination with reducing polysaccharides can be increased by introducing aldehyde groups which do not cyclize to hemiacetals. This can be achieved by reducing the reducing end to the sugar alcohol, followed by selective oxidation of the vicinal diols in the opened sugar alcohol (Y. C. Lee, R. T. Lee, Neoglycoproteins: Preparation and Application, Academic Press, San Diego 1994) (FIG. 2.1d).

Besides the direct coupling of the aldehyde-modified polysaccharides to amino functions of the protein by reductive amination, it is also possible in this way to modify the polysaccharide with bifunctional hydrazide linkers (see below).

Introduction of Amino Functions

Compared with polysaccharides, the possibilities of reacting the reducing sugar by a reductive amination to give glycamines or to give glycosylamines with intact ring structure are better in the case of oligosaccharides (with up to 20 carbohydrate monomers) because the reactivity is somewhat higher (FIG. 2.2).

The use of a bifunctional linker is appropriate for coupling the amino-modified polysaccharides to the various side-chain functions of the protein (see below).

Introduction of Amino Functions by Reductive Amination

In contrast to the glycamine synthesis by reductive amination with NH₃ or aliphatic amines (B. Kuberan et al., Glycoconj. J. 1999, 16, 271-81), higher yields can be achieved with aromatic amines such as, for example, benzylamine (T. Yoshide, Methods of Enzymol. 1994, 247, 55-64), 2-(4-aminophenyl)ethylamine (APEA) (H. D. Grimmecke, H. Brade, Glycoconj. J. 1998, 15, 555-62) or 4-trifluoroacetamidoaniline (E. Kallin, Methods Enzymol. 1994, 247, 119-23) under comparable conditions (FIG. 2.2a).

Whereas in the case of APEA the difference in the reactivity of the aliphatic and aromatic amino functions is exploited for a selective reaction, a monoprotected compound is available in the form of 4-trifluoroacetamidoaniline (alternatively, benzyloxycarbonylaminoaniline is also employed (M. Barström et al., Carbohydr. Res. 2000, 328, 525-31)), subsequent elimination of the trifluoroacetyl group in turn liberating an aromatic amino function. It has additionally emerged that glycamines can be stabilized by simple N-acetylation with acetic anhydride before elimination of the TFA protective group.

Introduction of Amino Functions by N-Glycosylation

N-Glycosilation (FIG. 2.2b) provides a possibility for retaining the cyclic ring structure of the reducing sugar. The unstable β-glycosylamines obtained by reaction with ammonium bicarbonate (I. D. Manger et al., Biochemistry 1992, 31, 10724-32; I. D. Manger et al., Biochemistry 1992, 31, 10733-40; S. Y. C. Wong et al., Biochem. J. 1993, 296, 817-25, E. Meinjohannes et al., J. Chem. Soc., Perkin Trans. 1, 1998, 549-60) can be stabilized by subsequent acylation with chloroacetic anhydride and be converted by aminolysis into the 1-N-glycyl compounds with free amino functionality. The N-glycosilation can be carried out analogously with allylamine and, after stabilization by N-acetylation, cysteamine can be added photochemically to the double bond (D. Ramos et al., Angew. Chem. 2000, 112, 406-8).

Preparation of Amino Functions from the Aldonic Acids

Free amino functions can be introduced by reaction with diamines into the aldonic acids which can be obtained by oxidation of reducing polysaccharides. This is possible through reaction of the acid with carbodiimides and diamines. Alternatively, the lactones which can be obtained by dehydration of the aldonic acids can be reacted with diamines (S. Frie, Diploma Thesis, Fachhochschule Hamburg, 1998).

Coupling Reactions of Modified HES and Allergenic Proteins using Bifunctional Linkers

The diversity of the functional groups of the modified HES and protein side chains which are to be connected together via a linker is paralleled by that of the available reaction possibilities (FIG. 3 shows common linker activations).

A distinction can be made for the reactive groups between reactivity towards amino groups (NHS esters, imido esters and aryl azides), aldehydes and (in the presence of EDC) carboxylic acids (hydrazides) or SH groups (maleimides, haloacetates or pyridyl disulfides).

Reagents with Amine Reactivity

The most useful coupling reagents are the amine-reactive crosslinkers. Moreover, the N-hydroxysuccinimide (NHS) esters (FIG. 3.1a) represent the commonest form of activation. In this case, the acylated compounds are formed by elimination of NHS. A further possibility for modifying primary amines is provided by the imido esters (F. C. Hartman, F. Wold, Biochemistry 1967, 6, 2439-48) (FIG. 3.1b), with imidoamides (amidines) being formed. The imido esters are frequently used as protein crosslinkers and are distinguished by minimal reactivity towards other nucleophiles. In addition, various aryl azide linkers are available (photoreactive crosslinkers), with which short-lived nitrenes are formed by photolysis. Dehydroazepines are produced therefrom by ring expansion (instead of a nonspecific insertion) and preferably react with nucleophiles, especially amines (FIG. 3.1c).

Because of the large number of commercially available coupling reagents with amino activity and variable linkers, other reaction possibilities such as, for example, reaction with isocyanates and isothiocyanates have increasingly lost importance.

Reagents with Reactivity Towards Carbonyl or Carboxyl Groups

Hydrazid linkers are used to couple compounds having carbonyl or carboxy groups (D. J. O'Shanessy, M. Wilchek, Anal. Biochem. 1990, 191, 1-8) (FIG. 3.2). Whereas aldehydes are converted to hydrazones which can be stabilized by reduction with NaCN/BH₃, carboxyl groups react in the presence of EDC to form imide linkages. The hydrazide-activated linkers represent a versatile alternative to reductive amination and to the carboxyl activations with zero-length crosslinkers such as carbonyldiimidazole (CDI).

Reagents with Sulfhydryl Reactivity

Coupling reagents with SH reactivity represent a second large class of crosslinkers. The coupling reactions primarily include two reaction pathways: alkylation (FIG. 3.3a-b) or disulfide exchange (FIG. 3.3c). Besides alkylation with α-haloacetates, the double bond of maleimides can be reacted selectively by Michael addition with SH groups to form a stable thioether linkage. The thiol-disulfide exchange represents a further sulfhydryl-specific reaction. In this case, reaction with pyridyl disulfides (J. Carlsson et al., Biochem. J. 1978, 173, 723-37) proves to be particularly advantageous because complete conversion to the mixed disulfides can be achieved by elimination of 2-pyridone.

Crosslinkers

The abovementioned coupling reactions by diverse homo- and heterobifunctional crosslinkers are used to synthesize the HAS-allergen bioconjugates of the invention.

Homobifunctional Crosslinkers

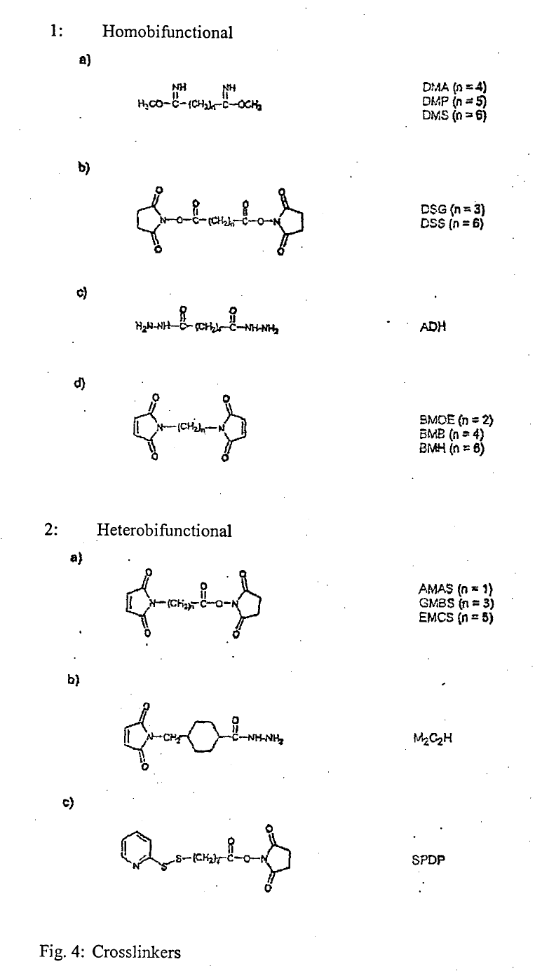

Symmetrical homobifunctional linkers (cf., for example, those depicted in FIG. 4.1) have the same reactive group at both ends and are suitable for linking together compounds having identical functional groups. According to the available coupling reactions, corresponding bifunctional linkers with, for example, bisimido esters, bissuccinimide, bishydrazide and bismaleimide functionalities are commercially available.

One disadvantage of the use of homobifunctional linkers is that crosslinking cannot be completely prevented in the activation of the first compound even on use of a large excess of crosslinker (S. Bystrick et al., Glycoconj. J. 1999, 16, 691-95). Complete removal thereof before the coupling to the second compound is necessary and may be difficult if the activated intermediate product is unstable (e.g. sensitivity of NHS-activated compounds to hydrolysis). Both amine reactivity and hydrolysis of NHS esters increase with increasing pH, which is why reactions are carried out under physiological conditions (pH 7) in buffered solutions (the half-life of the NHS ester DSP at 0° C. and pH 7 is 4-5 hours, but is only 10 min at pH 8.6; A. J. Lomant, G. Fairbanks, J. Mol. Biol. 1976, 104, 243-261).

Heterobifunctional Crosslinkers

Heterobifunctional coupling reagents (cf., for example, those depicted in FIG. 4.2) can be used to link together compounds having different functional groups. The linkers are provided with two different reactive groups and, by combining different coupling reactions, can be reacted selectively at one end of the crosslinker. Thus, for example, one side of the linker has amino activity and the other has sulfhydryl activity, resulting in a better possibility of reaction control compared with homobifunctional linkers.

The more reactive or more unstable side of the heterobifunctional linker is reacted first. Since NHS esters can react not only with amino groups to form a stable amide linkage, but also with sulfhydryl and hydroxyl groups, the heterobifunctional linker is initially reacted with the amino compound. In relation thereto, the maleimido group shows not only greater selectivity but also a greater stability in aqueous solution, so that the activated intermediate can be purified and subsequently reacted selectively with the compound having sulfhydryl activity.

The choice of the crosslinker depends not only on the nature of the functional groups used for the coupling, but also on the desired length and composition, called the cross-bridge, of the spacer. Thus, some spacers, especially those having rigid ring structure such as, for example, SMCC or MBS, elicit a specific antibody response (J. M. Peeters et al., J. Immunol. Methods 1989, 120, 133-43) and may thus be less suitable for hapten-carrier immunogens and in vivo use.

The compilation of linkers in FIG. 4 omits the specifically cleavable linkers which can be opened by disulfide cleavage (e.g. DSP, DTME or DTBP) or periodate cleavage (diols such as BMDB or DET) and are used to study biospecific interactions or for purifying unknown target structures.

The abbreviations used for the commercially available coupling reagents are derived from the systematic names of the compounds, such as, for example, DMA (dimethyl adipimidate), DMS (dimethyl suberimidate), GMBS (N-(γ-maleimidobutyryloxy)succinimide ester) etc.

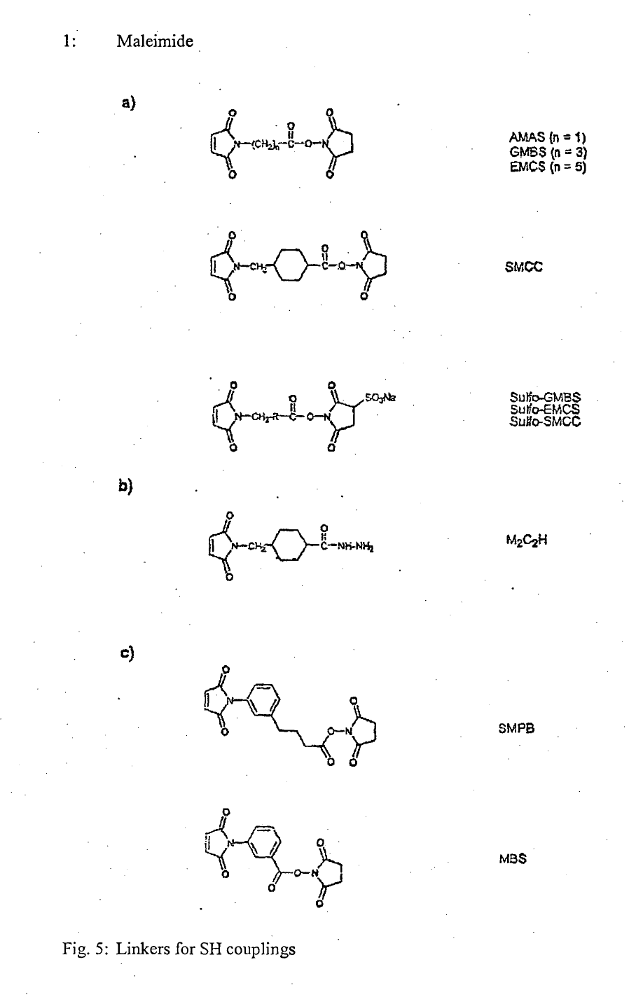

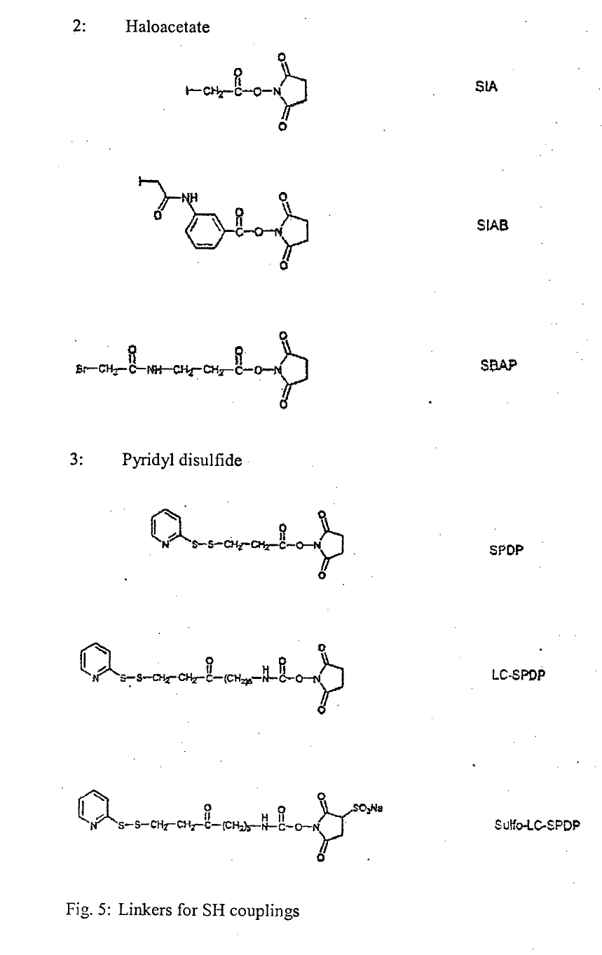

An overview of various heterobifunctional crosslinkers which could be used for example for sulfhydryl couplings is shown in FIG. 5.

The greatest versatility is provided here by the maleimide-activated linkers, usually combined with NHS ester activation. These linkers with sulfhydryl and amino reactivity are water-insoluble, linear alkyl-bridged linkers such as, for example, AMAS, GMBS and EMCS or have, like SMCC, SMPB or MBS, a rigid ring structure. The two UV active linkers SMPB and MBS are normally used for immunochemical methods such as ELISA assays.

In addition, M₂C₂H is a linker with the same rigid bridging as in SMCC but with hydrazide activation for linkage of compounds having sulfhydryl and carbonyl or carboxyl activity.

In contrast to the water-insoluble linkers, which need to be dissolved firstly in an organic solvent such as DMF or DMSO before the reaction, the water-soluble variants of some linkers are additionally available as the hydrophilic sulfo-NHS esters (J. V. Staros, Biochemistry 1982, 21, 3950-55), such as, for example, sulfo-GMBS, sulfo-EMCS and sulfo-SMCC.

Besides the maleimide-activated heterobifunctional linkers, it is also possible to use for sulfhydryl couplings various haloacetates such as, for example, SIA (and the bromo analog), SIAB and SBAP (FIG. 5.2), and pyridyl disulfides such as SPDP and LC-SPDP and sulfo-LC-SPDP (FIG. 5.3), once again combined with an NHS ester activation for amino coupling. Haloacetates can be introduced into aminated polysaccharides also by reaction with the free acid and water-soluble carbodiimide (N. J. Davies, S. L. Flitsch, Tetrahedron Lett. 1991, 32, 6793-6796) or with the corresponding anhydride (I. D. Manger et al., Biochemistry 1992, 31, 10733-40; S. Y. C. Wong et al., Biochem. J. 1994, 300, 843-850) (cf. FIG. 2.2b).

Various examples of the coupling of synthetic oligosaccharides to SH side chains of proteins via heterobifunctional maleimide linkers are to be found in the literature (V. Femandez-Santana et al., Glycoconj. J. 1998, 15, 549-53; G. Ragupathi et al., Glycoconj. J. 1998, 15, 217-21; W. Zou et al., Glycoconj. J. 1999, 16, 507-15; R. Gonzalez-Lio, J. Thiem, Carbohydr. Res. 1999, 317, 180-90). In addition, direct couplings of iodoacetamide derivatives of oligosaccharides are also used for the specific glycosylation of proteins (N. J. Davies, S. L. Flitsch, Tetrahedron Lett. 1991, 32, 6793-679645; S. Y. C. Wong et al., Biochem. J. 1994, 300, 843-850).

Modification of Glycoproteins on the Glyco Moiety with Poly- and Oligosaccharides:

In the case of glycoproteins, the linked oligosaccharides also provide further linkage points to form the conjugates of the invention as alternative to the amino acid side chains of the protein (J. J. Zara et al., Anal. Biochem. 1991, 194, 156-62).

Introduction of Aldehydes by Oxidation with Sodium Periodate

Aldehydes can be introduced into non-reducing oligosaccharides by oxidation with sodium periodate. Depending on the chosen oxidation conditions, there can be selective oxidation of sialic acids present or less selective oxidation also of fucose, mannose, galactose and N-acetyl glucosamine residues (S. M. Chamov et al., J. Biol. Chem. 1992, 267, 15916-22). A possible side reaction is the formation of aldehydes from N-terminal serine, cysteine or threonine (D. J. O'Shanessy, M. Wilchek, Anal. Biochem. 1990, 191, 1-8).

Enzymatic Introduction of Aldehydes

Oxidation of glycoproteins with galactose oxidase leads to the formation of C6 aldehydes at terminal galactoses or N-acetylgalactosamines. However, these sugars are not terminal in particular in glycoproteins from animal cells, so that they must first be made available in a preceding step (D. J. O'Shanessy, M. Wilchek, Anal. Biochem. 1990, 191, 1-8).

Pharmaceutical Compositions

The present invention finally relates to pharmaceutical compositions which comprise an HAS-allergen conjugate of the invention. The conjugates of the invention are particularly advantageously suitable for producing pharmaceutical compositions which can be employed for the hyposensitization of allergy sufferers. The pharmaceutical compositions are particularly suitable for the therapy of allergy sufferers in whom an IgE-mediated sensitization has been detected and corresponding clinical symptoms have been observed.

Accordingly, the conjugates of the invention can be used in particular for producing pharmaceutical compositions which are suitable for the specific immunotherapy of patients with clinically relevant reactions to immediate-type allergens, such as, for example, people allergic to pollen, mites, mammalian hair (saliva), fungi, insects, foods and natural rubber/latex. The immunotherapy is thus particularly suitable for the treatment of asthmatics and hay-fever patients.

The compositions of the invention can be employed in various forms of specific immunotherapy, especially hyposensitization. Thus, the hyposensitization can be carried out by subcutaneous, mucosal, oral, peroral or sublingual administration of the HES conjugates of the invention. The hyposensitization can also be carried out in the form of various treatment protocols (preseasonal/perennial).

It may be appropriate in particular for people allergic to insects for the therapy to be carried out by the rush or ultra-rush method (cf. Kleine-Tebbe et al., Pneumologie, Vol. 5 (2001), 438-444).

The pharmaceutical compositions are produced by mixing the conjugates of the invention with carriers and/or excipients which are suitable for the hyposensitization.

Conjugate of HES and Allergenic Glycoprotein

Examples of the types of chemical functionalities of the glycoprotein which can be used for the coupling to prepare HES-glycoprotein conjugates are the following:

A: the thiol group of a cysteine side chain

B: the aldehyde group of an oxidized galactose residue.

Accordingly, alternative B does not apply to proteins which are not glycolized.

HES is distinguished by a single reductive end. Because of this structural feature, HES is particularly suitable for targeted regioselective linkage for the purposes of the present invention.

Approaches to chemical ligation which can be employed for the HES-protein conjugate synthesis are those developed for constructing larger proteins from unprotected peptide fragments. These approaches are based on the choice of in each case unique reactive functions in the fragments to be linked, which react selectively with one another to give a stable end product in the presence of the large number of other functions in natural proteins.

The HES preparation will generally be converted firstly into a defined, highly purified and well characterized intermediate (reactive HES) which can then react spontaneously and regioselectively under physiological conditions with the target function of the allergen.

Selective conversion of the reductive end of HES into a primary amino function (1-amino-HES) is preferred. This “1-amino-HES” can then be flexibly adapted to the linkage reaction with the protein, it being possible to follow various synthetic routes and to combine reaction steps into one step through prefabricated reagents (linkers).

HS-Reactive HES

Alternative processes for preparing HS-reactive HES are described schematically and assessed below:
1.—reductive amination of HES with the bifunctional linker M₂C₂H (FIG. 5.1.b) to give HS-reactive HES (A);
   purification by dialysis and freeze drying;
   coupling of the HS-protein by Michael addition.
   This synthesis has particular advantages because it is very simple (1 step) and the reaction with the target protein takes place very selectively. If problems arise through the toxicity of hydrazine derivatives, they must subsequently be purified by purification processes known the art.
2.—reaction of the HES lactone (oxidized HES) with the bifunctional linker M₂C₂H (FIG. 5.1.b) to give HS-reactive HES (B);
   purification by dialysis and freeze drying;
   coupling of the HS-protein by Michael addition.
   This reaction differs from that described above under 1. through additional effort for preparing the HES lactone.
3.—reaction of HES with ammonium bicarbonate to give 1-amino-HES (C);
   purification by freeze drying;
   acylation of the 1-aminal with bromo/iodoacetic anhydride without base catalysis to give the bromo/iodoacetamide (HS-reactive HES D);
   purification by dialysis and freeze drying; coupling with HS-protein by alkylation.
   This process is also advantageous; it comprises only two steps and uses only very simple reagents. The process is therefore very cost effective. The scale of the synthesis can easily be expanded. The reaction with the target protein is very selective.
4.—reaction of the HES lactone (oxidized HES) with a diamine (1,4-diaminobutane) as described by Frie (S. Frie, Diplomarbeit, Fachhochschule Hamburg, 1998) to give an amino-HES (E);
   acylation of the amino-HES with bromo/iodoacetic anhydride without base catalysis to give the bromo/iodoacetamide (HS-reactive HES F);
   purification by dialysis and freeze drying; coupling with HS-protein by alkylation.
   This synthetic route differs from that described above under 3. through additional effort for preparing the HES lactone.
      CHO-Reactive HES

Alternative processes for preparing CHO-reactive HES are described schematically and assessed below:
1.—use of amino-HES (E) as CHO-reactive HES G;
   coupling with CHO-protein by reductive amination.
   This synthesis is very simple and cost effective. Competition from internal lysines may where appropriate cause problems which can be controlled through the choice of the reaction conditions.
2.—reaction of the HAS lactone (oxidized HES) with hydrazine to give the hydrazide (CHO-reactive HES H);
   purification by dialysis and freeze drying;
   coupling with CHO-protein by hydrazone formation at pH 5-6; the coupling reaction ought preferably to be carried out in situ during the oxidative formation (enzymatic or chemical) from galactose residues; a subsequent reductive stabilization with NaCN/BH₃ is optionally carried out;
   an enzymatic oxidation of the galactoses should preferably be carried out with a polymer-bound enzyme in order to facilitate removal of the enzyme.
   This synthesis is very simple and selective (no competition from internal lysines). Problems might arise owing to the toxicity of the hydrazine derivatives.
3.—further reaction of D or F with ammonium bicarbonate to give the glycinamides (CHO-reactive HES H and I);
   purification by dialysis and freeze drying;
   coupling with CHO-protein by reductive amination.
   This process takes place in three steps but uses very simple reagents and is thus cost effective. The scale of the synthesis can easily be expanded. However, competition from internal lysines might occur (cf. above).
4.—acylation of the amino-HES C or E with cBz-aminooxyacetic acid with subsequent hydrogenation to aminooxy-HES (CHO-reactive HES K);
   coupling with CHO-protein by oxime formation at pH 5-6; the coupling reaction should preferably take place in situ during the oxidative formation (enzymatic or chemical) from galactose residues;
   an enzymatic oxidation of the galactoses should preferably be carried out with a polymer-bound enzyme in order to facilitate removal of the enzyme.
   This synthesis is elaborate, but the coupling with the target protein is just as selective as in the reaction described under 2. (no competition from internal lysines).

1. The use of a conjugate of hydroxyalkylstarch and an allergen in which at least one hydroxyalkylstarch is covalently coupled to the allergen for hyposensitization.

2. The use as claimed in claim 1, where the hydroxyalkylstarch is coupled directly or via a linker to the allergen.

3. The use as claimed in claim 1, where the hydroxyalkylstarch is hydroxyethylstarch, hydroxypropylstarch or hydroxybutylstarch.

4. The use as claimed in claim 1, in which the hydroxyethylstarch has an average molecular weight of from 1 to 300 kDa.

5. The use as claimed in claim 1, in which the hydroxyethyl starch has a level of molar substitution of from 0.1 to 0.8 and a C2:C6 substitution ratio in the range from 2 to 20, in each case based on the hydroxyethyl groups.

6. The use as claimed in claim 1, in which the allergen has been selected from the group consisting of polypeptides or proteins.

7. The use as claimed in claim 1, in which the allergen is a glycoprotein.

8. The use as claimed in claim 1, in which the hydroxyalkylstarch is coupled to the polypeptide chain or to one or more of the saccharide chains of the glycoprotein.

9. The use according to claim 1 for hyposensitization of allergy sufferers in whom an IgE-mediated sensitization is detected or whose clinical symptoms have been observed.

10. The use as claimed in claim 1, where the specific immunotherapy is employed for the therapy of allergies to pollen, mites, mammalian hair (saliva), fungi, insects, foods and natural rubber/latex.

11. The use as claimed in claim 1, where the therapy is employed for the treatment of asthmatics, hay-fever patients and patients showing other types of clinically relevant reactions to immediate-type allergens.

12. The use as claimed in claim 1, where administration takes place subcutaneously, mucosally, orally, perorally or sublingually.

13. The use as claimed in claim 1, where the immunotherapy is carried out preseasonally or perennially for airborne allergens.

14. The use as claimed in claim 1, where the immunotherapy is carried out for people allergic to insects in the rush or ultra-rush method.

15. The use as claimed in claim 1, in which the hydroxyethylstarch has an average molecular weight of from 5 to 200 kDa
